(19) United States
(12) Patent Application Publication
(10) Pub. No.: US 2011/0177576 A1
(43) Pub. Date: Jul. 21, 2011

(54) SHOCK WAVE CELL TREATMENT DEVICE AND METHOD TO ENHANCE CELL REPLICATION

(75) Inventors: Wolfgang Schaden, Vienna (AT); Reiner Schultheiss, Illighausen (CH); Robert Goeschl, Winzendorf (AT); Johannes Holfeld, Vienna (AT)

(73) Assignee: TISSUE REGENERATIONTECHNOLOGIES, LLC., Woodstock, GA (US)

(21) Appl. No.: 13/009,145

(22) Filed: Jan. 19, 2011

Related U.S. Application Data
(60) Provisional application No. 61/297,018, filed on Jan. 21, 2010.

(51) Int. Cl. C12N 13/00; C12M 1/42

(52) U.S. Cl. 435/173.1; 435/289.1; 435/286.1

(57) ABSTRACT

A device 100 for treating cells or cultures of cells comprises: a fluid holding tank 100T, having sides or walls 101, 102, 103, 104 and a bottom 105, with an opening 39 in a wall or side for receiving an applicator 200M for transmitting energy into a holding tank 100T and an adapter 40 for insertion into the opening 39, the adapter being for attachment to the applicator 200M for transmitting energy into holding tank 100T, the adapter 40 forms a fluid tight seal between the opening 39 and the adapter 40, wherein the adapter 40 receives and holds a lens or diaphragm 201 through which the transmission of energy, preferably in the form of acoustic waves, passes.

### RELATED APPLICATIONS

This is a non-provisional application claiming benefit of priority to provisional application U.S. 61/297,018 entitled “Shock Wave Cell Treatment Device and Method to Enhance Cell Replication” filed Jan. 21, 2010.

### TECHNICAL FIELD

This invention relates to treating or stimulating cells held in a culture container or flask with acoustic shock waves. More specifically to a device for holding a container or flask of living cells in a fluid bath and a shock wave applicator for directing acoustic shock waves onto and through the held container to treat or otherwise stimulate the living cells while avoiding cellular damage due to unwanted reflected or defracted wave propagation.

### BACKGROUND OF THE INVENTION

Acoustic shock waves have been used in the field of medical treatments such as lipotripsy where focused high energy waves are directed to break up concrements.

As the science of using acoustic shock waves is rapidly advancing, variations in wave forms, energy levels and dosages have led to remarkable advances in treating patients with a variety of tissue related issues as is described in U.S. Pat. No. 7,470,240 B2.

Acoustic shock waves have been found to be useful in treating bones, ligaments, organs (such as the heart), nerves and wounds in patients. The shock waves have been credited with causing more rapid healing by increasing tissue regeneration and growth. Almost all these treatments have been directed to in vitro treatments of a damaged or injured region of a patient.

In US 2009/0254007 A1, the use of low energy acoustic shock waves to treat a culture of stem cells is disclosed to enhance replication and growth of the culture. In this publication it is stated “The use of shock waves as described above appears to involve factors such as thermal heating, light emission, electromagnetic field exposure, chemical releases in the cells as well as a microbiological response within the cells. Which combination of these factors plays a role in stimulating healing is not yet resolved. However, there appears to be a commonality in the fact that growth factors are released which applicants find indicative that otherwise dormant cells within the tissue appear to be activated which leads to the remarkable ability of the targeted organ or tissue to generate new growth or to regenerate weakened vascular networks in for example the cardio vascular system. This finding leads to a complimentary use of shock wave therapy in combination with stem cell therapies that effectively activate or trigger stem cells to more rapidly replicate enhancing the ability to harvest and culture more viable cells from the placenta, a nutrient culture of said stem cells, or other sources. The ability to stimulate stem cells can occur within the patients own body activating the naturally occurring stem cells or stem cells that have been introduced to the patient as part of a treatment beneficially utilizing stem cells. This is a significant clinical value in its own right.”

In studying the various methods to enhance the harvesting of stem cells, scientists have tried a variety of research techniques to grow stem cells in a culture. US 2009/0311735 teaches using a positively charged support structure. Similarly WO 2009/116951 teaches the use of a positively charged support matrix to promote the growth of stem cell cultures. WO 209117098 suggests using a microgravity condition to enhance growth. WO 2009150051 suggests employing a biocompatible material to promote growth of undifferentiated pluripotent stem cells.

With the exception of US 2009/0254007, all of these techniques for enhancing cell replication and growth deal with the environment of the culture or the physical support structure. All try to avoid contamination of the stem cell due to feeder cells.

The one statement in US 2009/0254007 relating to the use of acoustic shock wave use to enhance replication, fails to teach how best to achieve the method from a practical physical perspective.

The present invention provides and teaches a novel practical solution to how one can treat cells with exposure to energy, preferably acoustic shock waves. It teaches a novel device for achieving this goal of enhanced cell replication as well as a novel method which is described as follows.

### SUMMARY OF INVENTION

A device for treating cells or cultures of cells comprises: a fluid holding tank, having sides or walls and a bottom, with an opening in a wall or side for receiving an applicator for transmitting energy into the holding tank and an adapter for insertion into the opening. The adapter is constructed for attachment to an applicator for transmitting energy into holding tank. The adapter forms a fluid tight seal between the opening and the adapter, wherein the adapter receives and holds a lens or diaphragm through which the transmission of energy, preferably in the form of acoustic waves passes.

The device further comprises a cell culture flask holding mechanism to which the cell culture flask can be secured wherein the cell culture flask holding mechanism includes a slotted bar; a flask holder and a locking screw. The screw attached to the flask holder can be moved in the slot to a predetermined distance fixing the distance between a flask and the opening. A heater for heating the fluid in the holding tank maybe used along with a temperature controller and temperature sensor for measuring the fluid temperature in the holding tank. The flask holder has a locking screw to secure a flask in the holder and the bar has a scale adjacent the slot to indicate the distance between a front face of a held flask and the opening.

The preferred device includes a power supply to power the heater; and a temperature controller connected to the power supply, heater and sensor to regulate the fluid temperature in the holding tank.

The device also includes a modified applicator attached to the adapter, the modified applicator transmits energy in the form of acoustic shock waves, ultrasound waves, or magnetic pulses into the holding tank to stimulate cells being cultured in the flask.

The acoustic shock waves can be focused waves, unfocused waves or low energy shock waves.

A method of treating cells comprises the steps of: placing a flask containing cells in a fluid filled holding tank having an opening for receiving an applicator for transmitting energy waves or pulses; activating the applicator transmitting energy to the flask to stimulate the cells; fixing the distance between the flask and the applicator to a predetermined distance prior to transmitting energy; and controlling the temperature of the fluid within the fluid filled holding tank.

### Definitions

A “curved emitter” is an emitter having a curved reflecting (or focusing) or emitting surface and includes, but is not limited to, emitters having ellipsoidal, parabolic, quasi parabolic (general paraboloid) or spherical reflector/reflecting or emitting elements. Curved emitters having a curved reflecting or focusing element generally produce waves having focused wave fronts, while curved emitters having a curved emitting surfaces generally produce wave having divergent wave fronts.

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different ways, for example: A focused wave will become divergent once it has passed through the focal point. Spherical waves are also included in this definition of divergent waves and have wave fronts with divergent characteristics.

“Extracorporeal” occurring or based outside the living body or plant structure.

A “generalized paraboloid” according to the present invention is also a three-dimensional bowl. In two dimensions (in Cartesian coordinates, x and y) the formula yⁿ=2px [with n being ≠2, but being greater than about 1,2 and smaller than 2, or greater than 2 but smaller than about 2,8]. In a generalized paraboloid, the characteristics of the wave fronts created by electrodes located within the generalized paraboloid may be corrected by the selection of (p (−z,+z)), with z being a measure for the burn down of an electrode, and n, so that phenomena including, but not limited to, burn down of the tip of an electrode (−z,+z) and/or disturbances caused by diffraction at the aperture of the paraboloid are compensated for.

A “paraboloid” according to the present invention is a three-dimensional reflecting bowl. In two dimensions (in Cartesian coordinates, x and y) the formula y²=2px, wherein p/2 is the distance of the focal point of the paraboloid from its apex, defines the paraboloid. Rotation of the two-dimensional figure defined by this formula around its longitudinal axis generates a de facto paraboloid.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plain waves. “Nearly plane” waves can be emitted by generators having pressure pulse/ shock wave generating elements with flat emitters or curved emitters. Curved emitters may comprise a generalized paraboloid that allows waves having nearly plane characteristics to be emitted.

A “pressure pulse” according to the present invention is an acoustic pulse which includes several cycles of positive and negative pressure. The amplitude of the positive part of such a cycle should be above 0.1 MPa and its time duration is from below a microsecond to about a second. Rise times of the positive part of the first pressure cycle may be in the range of nano-seconds (ns) up to milli-seconds (ms). Very fast pressure pulses are called shock waves. Shock waves used in medical applications do have amplitudes above 0.1 MPa and rise times of the amplitude are below 100 ns. The duration of a shock wave is typically below 3 micro-seconds (μs) for the positive part of a cycle and typically above 3 micro-seconds for the negative part of a cycle.

‘Waves/wave fronts” described as being “focused” or “having focusing characteristics” means in the context of the present invention that the respective waves or wave fronts are traveling and increase their amplitude in direction of the focal point. Per definition the energy of the wave will be at a maximum in the focal point or, if there is a focal shift in this point, the energy is at a maximum near the geometrical focal point. Both the maximum energy and the maximal pressure amplitude may be used to define the focal point.

### BRIEF DESCRIPTION OF THE DRAWINGS

The invention will be described by way of example and with reference to the accompanying drawings in which:

FIG. 1 is a perspective view of the fluid and culture holding device made in accordance to the present invention.

FIG. 2 is a top view of the culture flask holder mechanism.

FIG. 3 is a side view of the culture flask holder mechanism.

FIG. 4 is an exploded view of the culture flask holder mechanism.

FIG. 5 is a perspective view of a shock wave applicator with the outer lens protective cover shown being removed.

FIG. 6 is the perspective view of FIG. 5 with the cover, ring lens or diaphragm holder, and the lens or diaphragm removed from the applicator.

FIG. 7 is a top perspective view of the applicator adapter.

FIG. 8 is a bottom perspective view of the applicator adapter.

FIG. 9 shows the applicator as modified with the lens or diaphragm being held in place by the applicator adapter attached to the applicator.

FIG. 10 is a side view of the applicator as modified in FIG. 9.

FIG. 11 is a perspective view of the modified applicator and the fluid and culture holding device.

FIG. 12 is a perspective view of a power supply unit.

FIG. 13 is a temperature control unit for plugging into the power supply unit.

FIG. 13A is a perspective view of the fluid and culture holding device with the modified applicator attached and a temperature sensor being installed.

FIG. 13B shows power supply lines being connected to a heater inside the holding device.

FIG. 14 shows a side view of the assembled device according to the present invention.

FIGS. 14A, 14B and 14C show the side view with various acoustic shock waves impinging the cell culture flask.

### DETAILED DESCRIPTION OF THE INVENTION

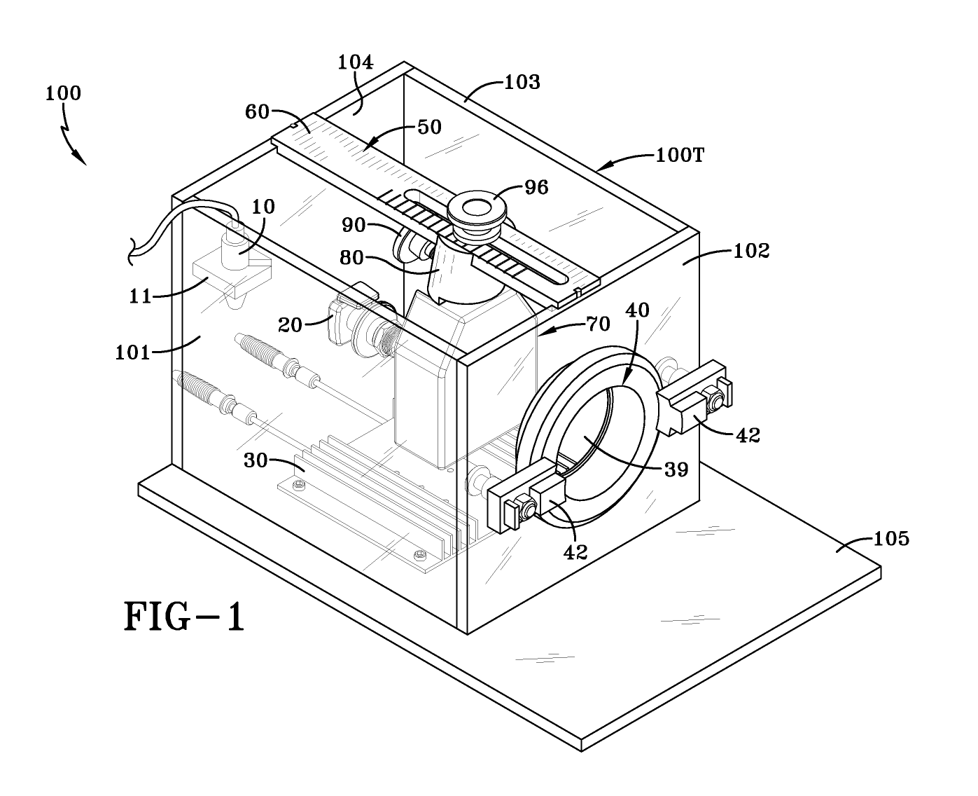

With reference to FIG. 1, the fluid 2 and culture flask 70 holding device 100 is shown. The device 100 has a fluid holding tank 100T which as shown is a four sided or walled tank with sides or walls 101, 102, 103 and 104 and a bottom 105, all made of clear plexiglass or a clear acrylic plastic material having an open top upon which rests a culture flask holding mechanism 50. The device 100 has an adapter opening 39 on one side 102 with a pair of safety catches 42 for securing an adapter 40 which is attached to a modified shock wave applicator 200M (not illustrated). Inside the tank 100T is a heater 30 connected to the bottom 105. A drain plug 20 is shown on a lower portion of a side 104 opposite the adapter opening 39, a temperature sensor 10 is held in the corner of the device 100 in a slotted bracket 11.

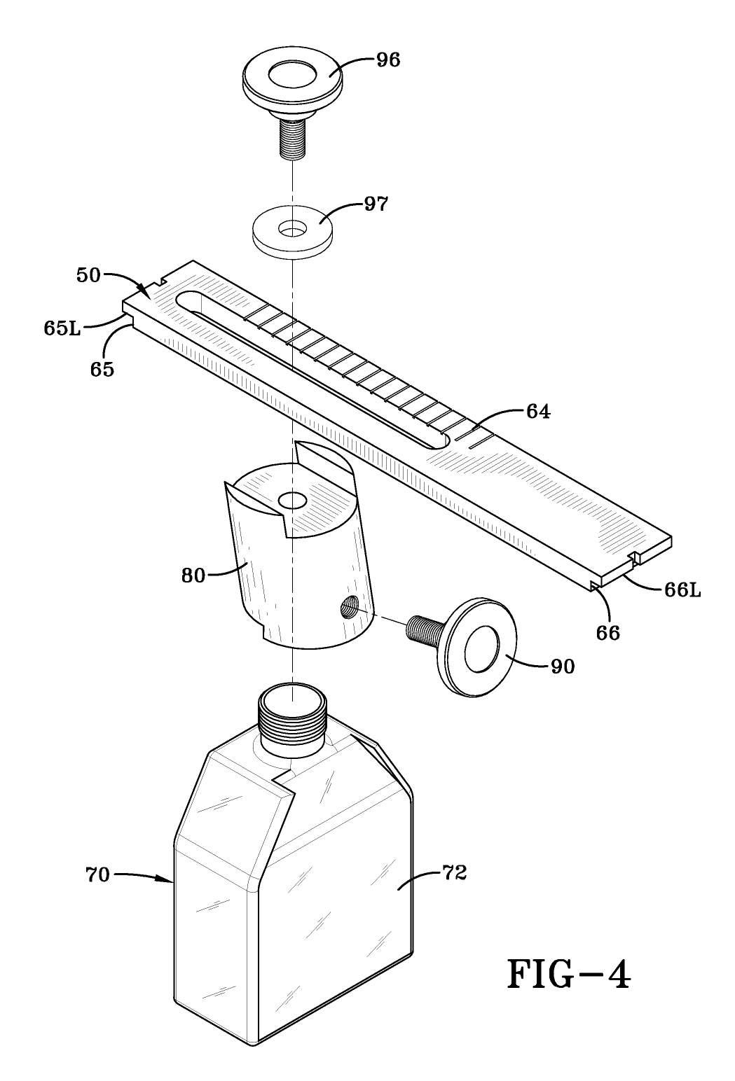

The culture flask holding mechanism 50 is shown in greater detail in FIGS. 2, 3 and 4. As shown the mechanism 50 includes a distance control bar 60. The control bar 60 has a slot 62 which allows a predetermined focal distance to be established from the lens of an applicator to the front facing side 72 of a culture flask 70. The threaded or open or capped end 73 of flask 70 is placed inside a flask holder 80. A locking screw 90 secures the threaded flask 70 in the holder 80. The screw 90 is screwed into the threads 81 of the holder 80 to hold the flask 70 in place. A second locking screw 96 and a washer 97 are placed over the bar. The threaded shank of the locking screw 96 is attached to the threaded hole 83 of the holder 80. This locks the flask 70 to the bar 60 setting or fixing the distance the flask 70 is from the applicator.

As shown in FIGS. 1 and 2, the bar 60 has a scale 64 marked onto a portion of the bar 60 adjacent the slot. The marks indicate the distance the front face 72 of the flask 70 is placed from the applicator lens or diaphragm and is used to permit the flask 70 to be set a predetermined distance in the transmitted acoustic shock wave. In this way the energy levels used to bombard the cells inside the flask 70 can be controlled.

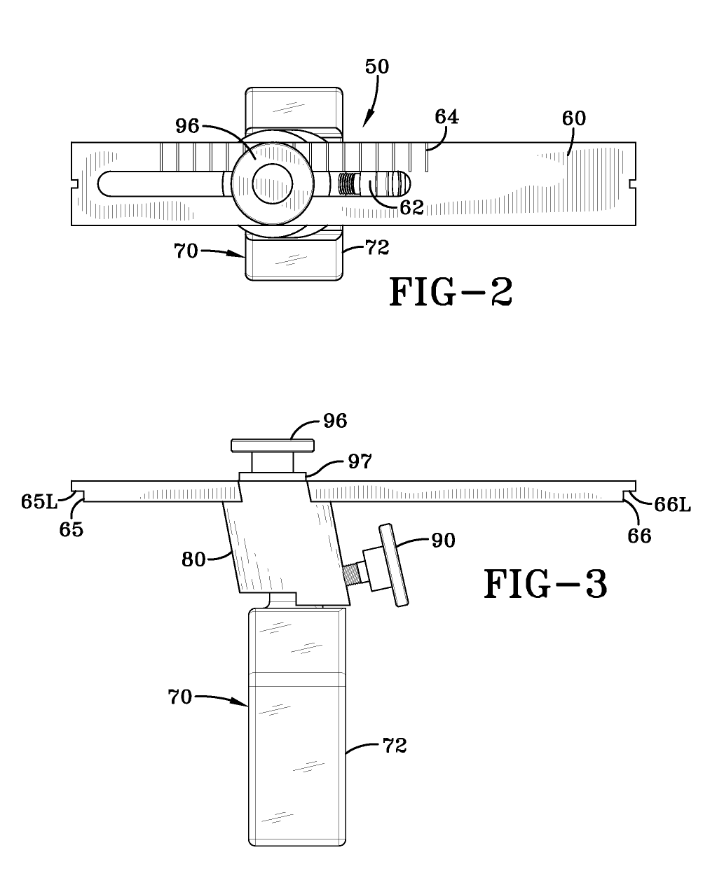

As shown in FIG. 3, the bar 60 has ends 65, 66 cut out to drop into the holding device and rest on ledges 65L, 66L which sit on the top of the sides 102, 104 in such a way that the bar 60 is fixed relative to the sides 102, 104 of the device 100. In this way the bar 60 holding the culture flask 70 is fixed so the screw 96 when moved in the slot 62 can precisely adjust the distance between the flask and the applicator so the transmitted shock waves can pass through the flask 70 at the desired energy level based on the distance from a wave focus of the beam.

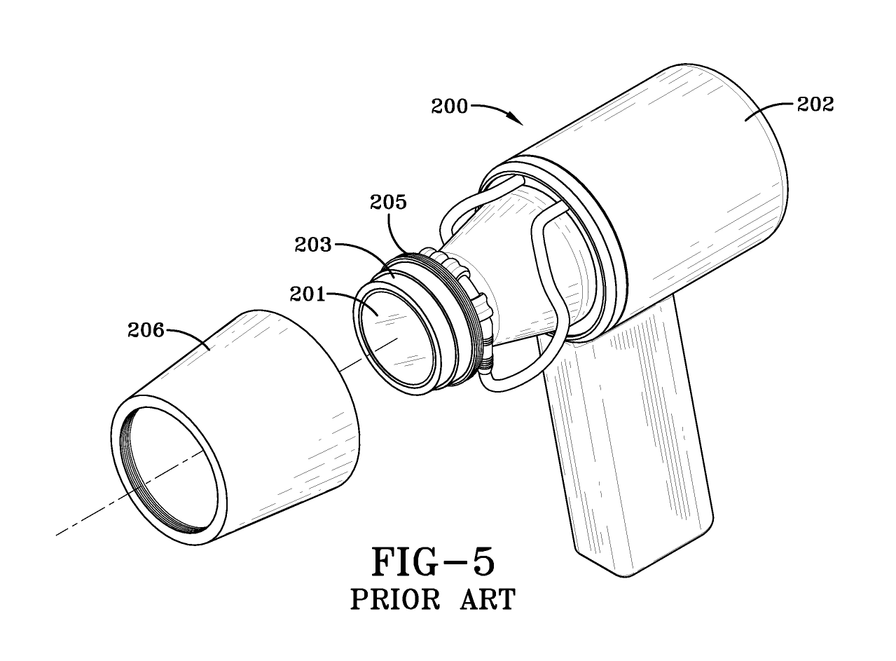

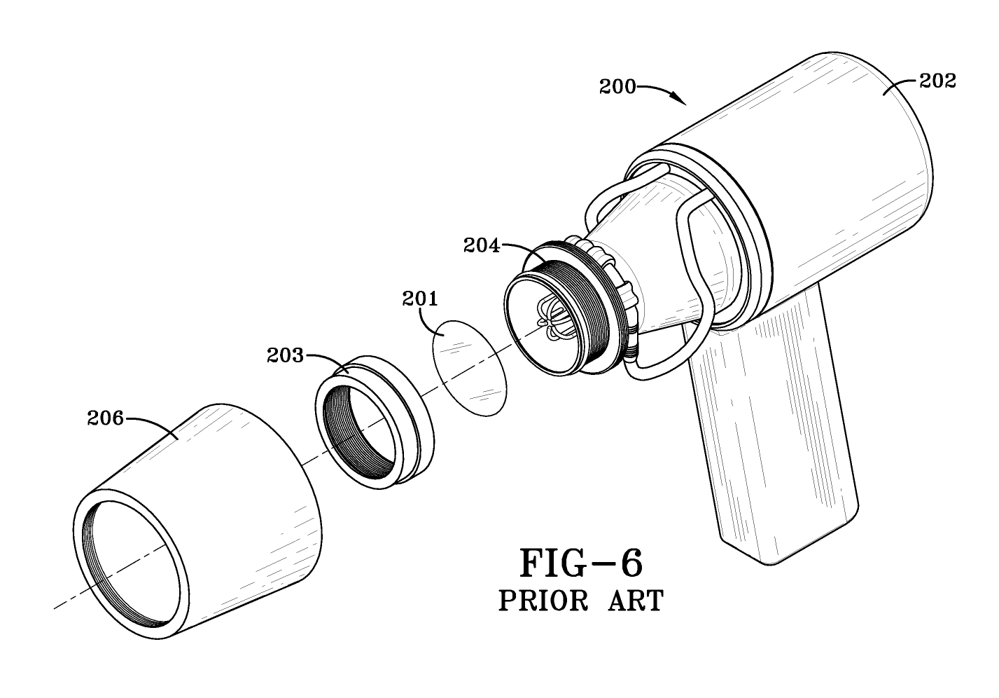

With reference to FIGS. 5 and 6, a prior art shock wave applicator 200 is illustrated. The shock wave applicator 200, as illustrated, has a body housing 202. As shown in FIG. 5 when the protective cover 206 is removed it exposes the lens or diaphragm 201, the lens holder adapter 203 and the threaded portion 205 of the applicator to which the cover 206 has been secured. As illustrated in FIG. 6 when these components are removed the applicator 200 with lens removed exposes threads 204 of the one end of the applicator 200.

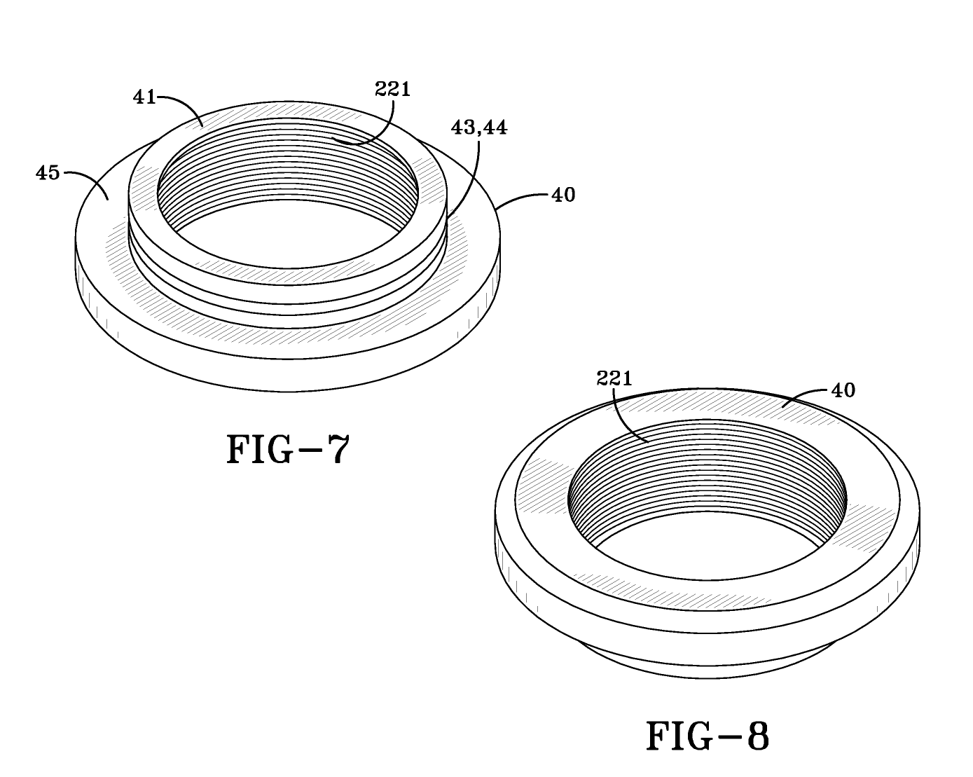

With reference to FIGS. 7 and 8, the adapter 40 is shown. The adapter 40 has a protruding cylindrical end 41 extending to a flange portion 45. Internally of the cylindrical end 41 are threads 221. These threads 221 are used to be attached directly to the applicator 200 at threads 204. On an exterior surface end 41of the adapter 40 is a groove 43 onto which an elastomeric O ring 44 is to be attached. This O ring 44 will provide a seal against the opening 39 in the side 102 when the applicator is mounted to the holding device 100. To facilitate insertion into the opening 39, grease or a lubricant can be applied to the O ring 44.

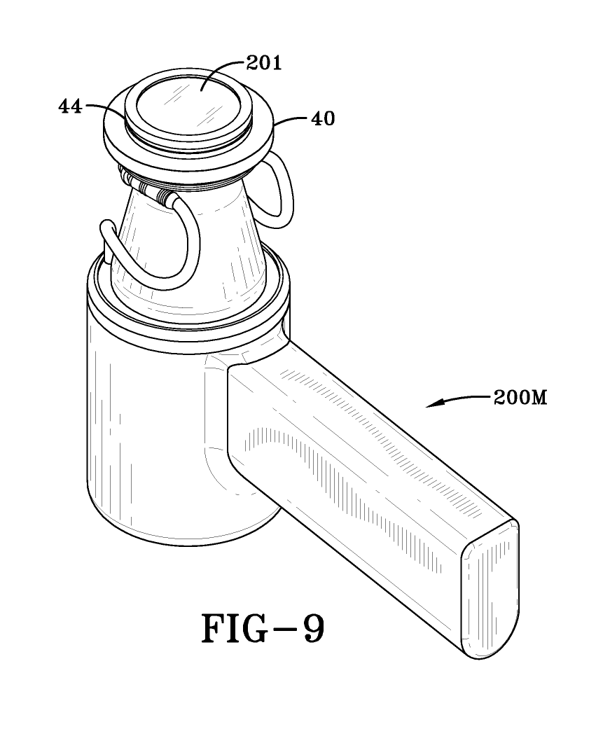

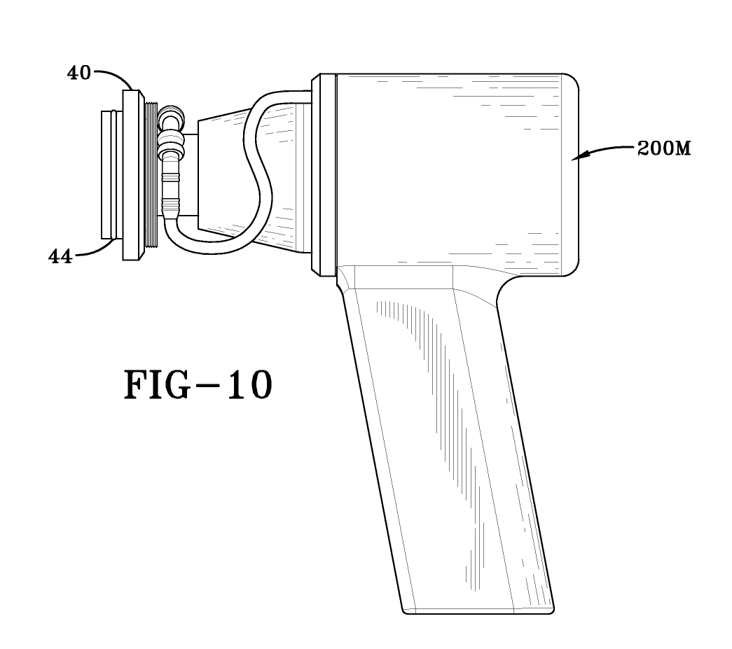

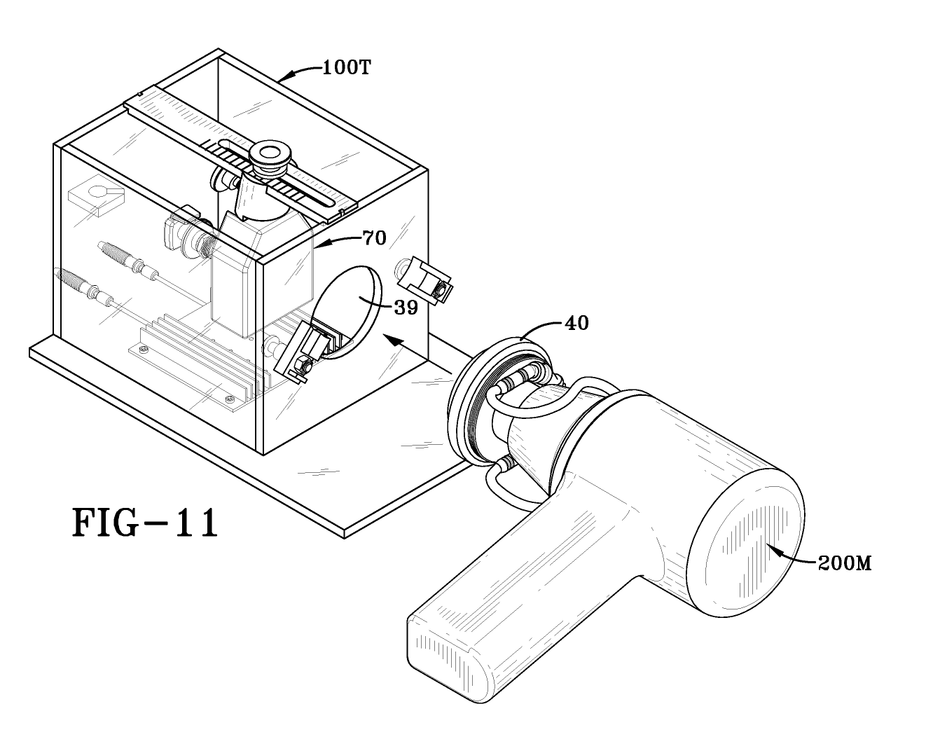

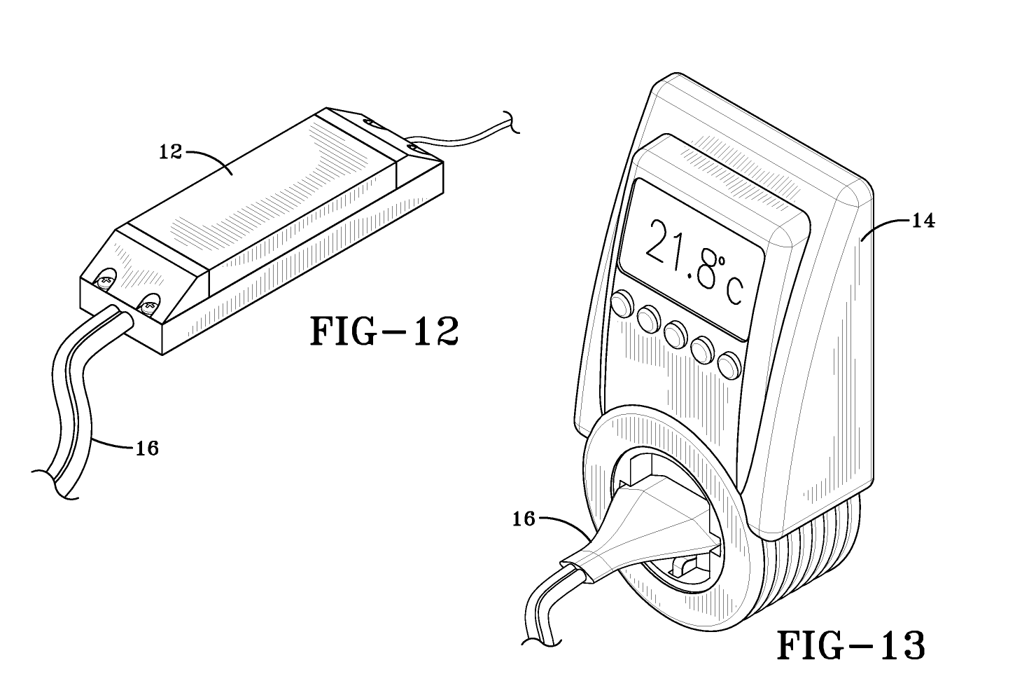

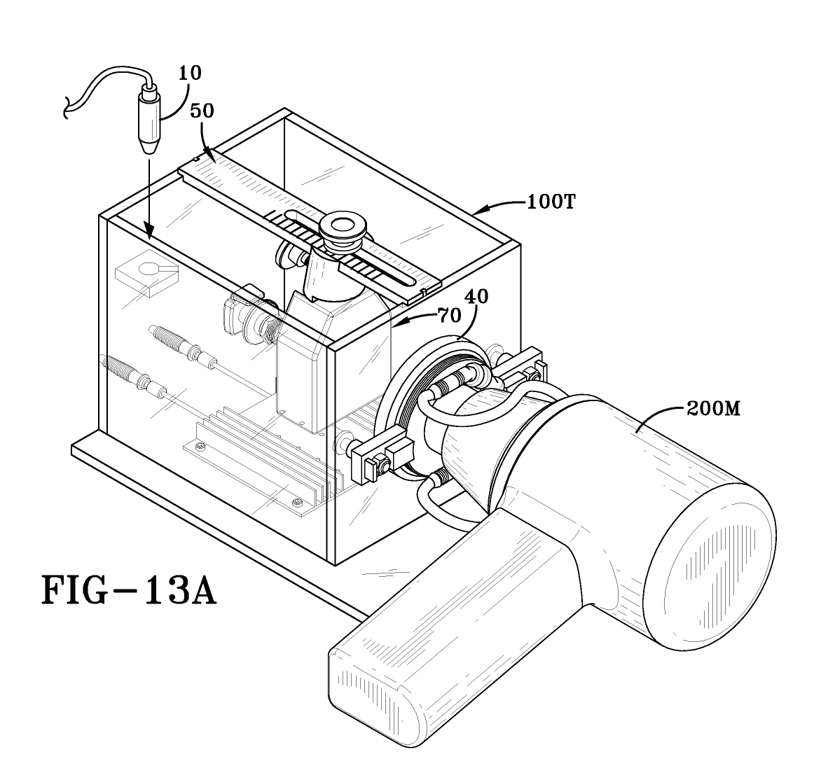

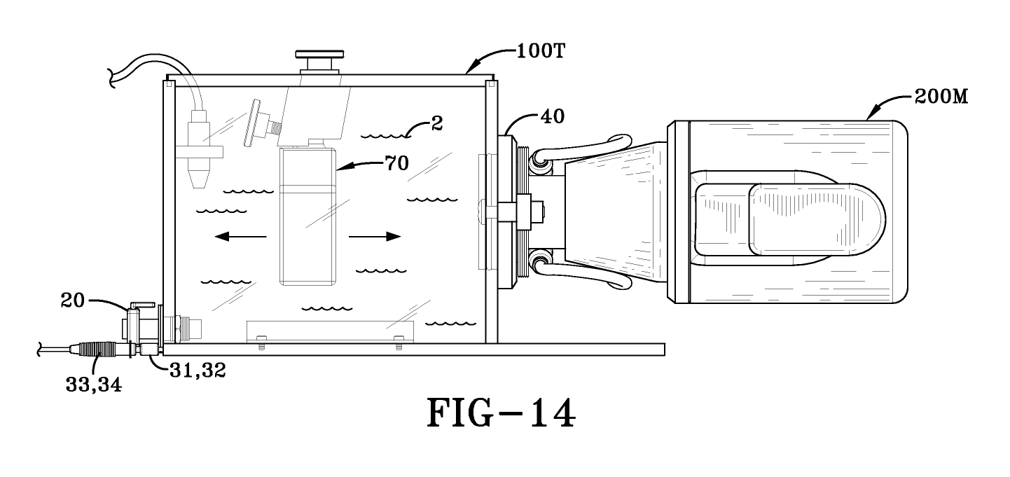

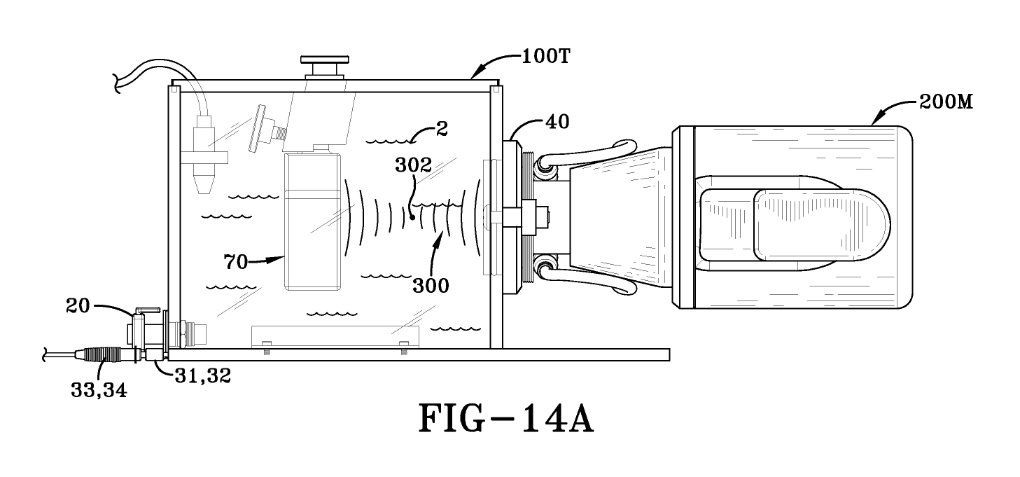

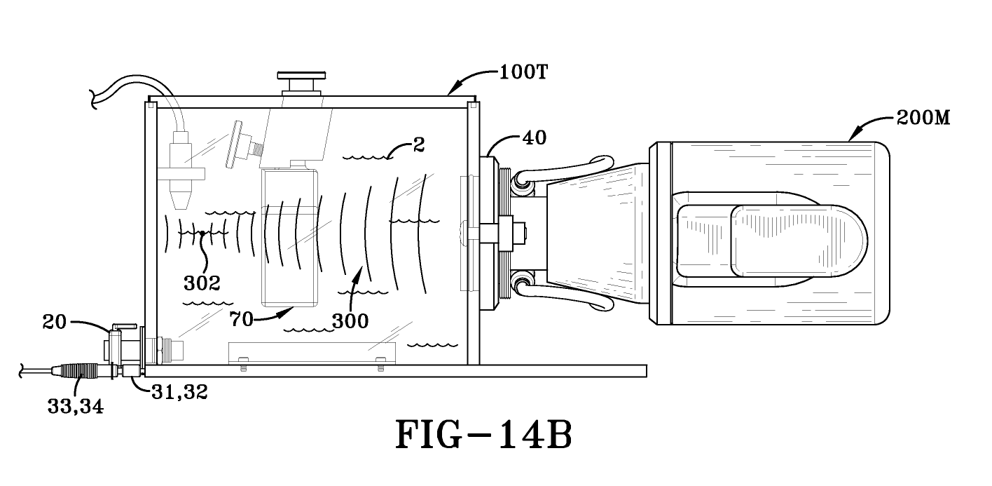

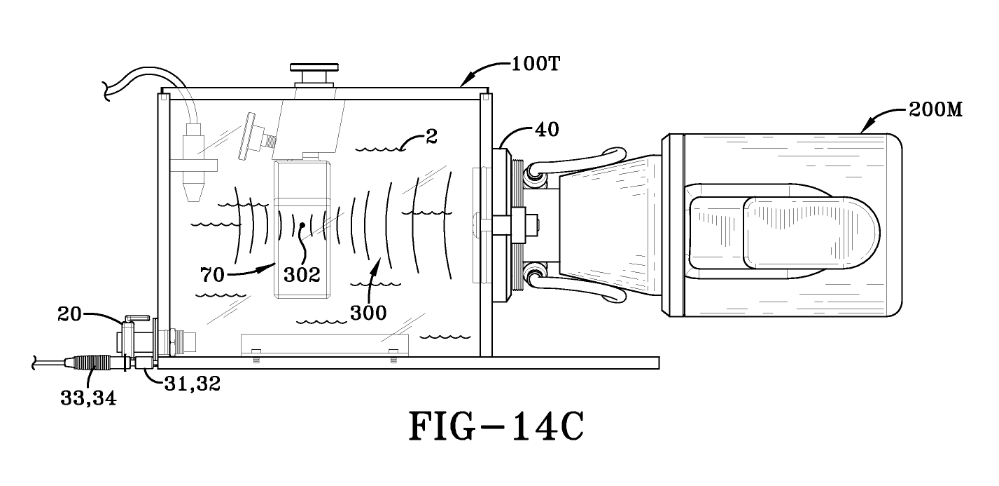

As illustrated in FIGS. 9 and 10, the adapter 40 when attached to the applicator 200 has the lens or diaphragm 201 installed and secured in such a way that it makes a water tight seal between the lens or diaphragm 201 and the adapter 40. When this assembly is completed as shown in FIGS. 9 and 10, the modified applicator 200M is formed being modified in such a way that the adapter 40 attached to the end of the applicator 200M can be inserted into the holding device 100 through the opening 39. Once assembled to the device 100 as illustrated in FIGS. 13A, 13B and 14, the assembly provides a water tight and hermetically sealed interface between the modified applicator 200M and the device 100 in such a way that energy, preferably shock waves, can be produced and transmitted that will impinge directly onto the cell culture holding flask 70. The distance between the lens 201 or the focal point 302 and the flask 70 can be fixed by using the thumb screw 96 and the bar 60 in such a way that the distance from the focal point 302 and the culture flask 70 is known and set.

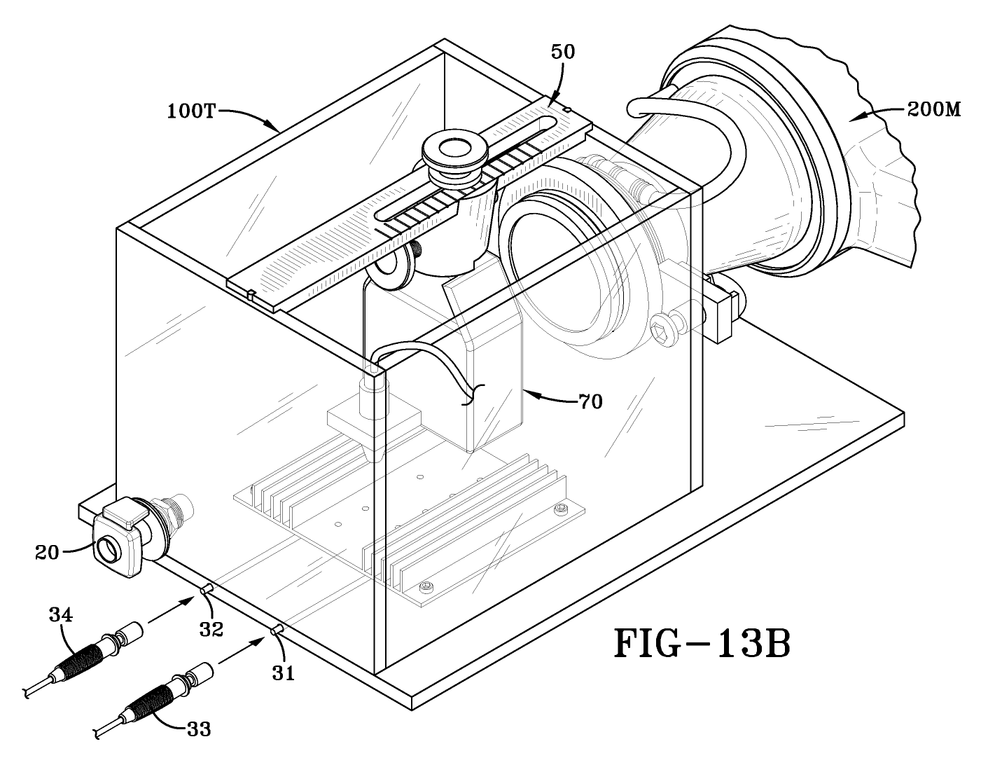

With further reference to FIGS. 12 and 13, a power supply unit 12 is provided which can be plugged into an alternating current device not shown. From the power supply unit 12 a temperature control device 14 can be connected through the cable 16. The temperature control device 14 is connected to the temperature sensor 10 which measures the fluid temperature in the tank 100T. The temperature control device 14 is connected electrically by the wire connectors 33, 34 which are connected to the heater 30 at the two electrical connections 31, 32 on the device 100 providing electrical power to the heater 30. Once this is accomplished temperature settings can be set on the temperature control device 14 to control the heater 30. Once the heater 30 heats fluid 2 in the device 100 to an optimal temperature, it is sensed by the sensor 10 shown in FIG. 1 and further heating can be reduced to maintain the holding tank temperature. Prior to initiating any heating of the holding tank device 100 the tank 100T is filled with water or suitable fluids 2 covering the flask 70 and the shock wave applicator lens 201 as illustrated in such a way that shock waves can be transmitted through the fluids 2 directly to the cell culture holding flask and pass outwardly therefrom.

One of the most intriguing acoustic shock wave treatments seems to be in the use of low energy acoustic shock waves to treat cells or cell cultures. These wave forms can pass through the cells without damaging the outer membrane or wall of the cell. This occurs as a result of the avoiding of cavitational effects in the cells or culture. The unfocused shock waves can be of a divergent wave pattern or near planar pattern preferably of a low peak pressure amplitude and density. Typically the energy density values range as low as 0.000001 mJ/mm² and having a high end energy density of below 1.0 mJ/mm², preferably 0.20 mJ/mm² or less. The peak pressure amplitude of the positive part of the cycle should be above 1.0 and its duration is below 3 microseconds, preferably below 1 microsecond. Similarly the focused waves can be in this energy range or higher if used pre or post convergence. That is to say if the cells are positioned before the waves converge to a focal point or after. Thus avoiding the peak energy locations. The shock waves can be focused low energy waves or unfocused.

With reference to FIGS. 14A, 14B and 14C, the device 100 is shown where the flask 70 is positioned relative to the wave form. As shown in FIG. 14A, the held flask 70 is positioned at a distance further back from the focal point 302 of the transmitted waves 300. By positioning the culture flask 70 at a distance away from this peak energy position the waves converge to a point then diverge as they move outward in the tank 100T toward the flask, as a result of this, when the acoustic wave impinges the cells within the flask 70 they are at the lower energy level thereby creating no possibility of damaging the cells being cultured within the flask 70. With reference to FIG. 14B, the flask 70 is shown positioned well ahead of the focal point 302. In this case the transmitted acoustic waves 300 are converging toward the focal point 302, but the flask 70 is positioned well in advance of the propagation of the waves in such a fashion that the cells again do not experience the peak energy levels at the focal point and thus can be safely treated with this acoustic wave without the possibility of damage. With reference to FIG. 14C, the device 100 is shown wherein the flask 70 is held at a position where the focal point 302 directly impinges inside the flask 70. Under these conditions, the converging waves reach a peak amplitude at the focal point 302, as a result, extreme care must be used when the flask 70 is in this position, as a result, much lower energy levels will be required to avoid cell damage. Another problem by positioning the flask 70 in this position is that the volumetric area of the cells being treated is quite limited as the wave pattern is reduced in size down as it converges to the smallest focal point and then must expand diverging as it leaves the flask 70.

While the present invention is capable of using any position for treating the culture cells, it is preferably that the flask 70 and the cells be positioned away from any high energy focal point and well within the diverging or converging portions of the wave forms. Alternatively, the wave transmissions can be such that planar waves or waves that do not converge can be transmitted in such a fashion that larger volumetric areas can be treated. Any of these possible combinations are possible, but the primary purpose of the device 100 is to enable the one trying to treat the cells to be able to move the flask 70 in a position most favourable for treatment regardless of the energy source, whether it be ultrasonic waves, acoustic shock waves, pulsed magnetic fields or light refraction.

Due to distracting physical effects that are associated with all existing shock wave in-vitro models, the device 100 forms an ideal water bath. The main improvement of this device 100 is the propagation of shock waves after passing the cell culture. In other models the difference of the impedance between culture medium and the ambient air causes reflections and diffraction.

Reflections disturb upcoming waves and even cause negative pressure onto the cell layer or floating cells. Mechanical forces are generated within the probe container if air bubbles are present, which is the case in most prior art experiments. These mechanical forces cause shear waves which disrupt cell membranes or compress cells completely that part of their structure is destroyed or worse total cell damage occurs. In cases when cells are attached to the wall or a surface the forces and the turbulences generated in the buffer or probe solution are sufficient to detach the cells of interest from the wall or surface impeding their growth. Therefore it is crucial to make shock waves propagate or otherwise converge after passing cell samples.

Minor reflections from the posterior wall of this water bath device 100 are comparable to those under in-vivo conditions, e.g. reflections from bones when treating muscles; therefore there is no need to inhibit this effect because the energy levels are so low. Alternatively if so desired, mounting a wave absorber to the back of the bath such as an air bladder could be done or extending the distance of the back of the device 100 relative to the flask 70 even further if higher energy transmissions are to be used to lower reflected energy.

The device 100 was primarily designed for basic research purposes to find better understanding about the shock waves molecular mechanism in cell cultures. The water bath device 100 now turns out to be a useful tool for all kind of cell and also tissue activating purposes. This includes three major fields of application; Tissue Engineering, Cell Activation/Treatment and Cell Membrane Permeation.

Tissue engineering: Tissue engineering means the colonization of cells on different scaffolds or matrices and also the forming of new tissue out of cell culture. Maybe someday even the engineering of complete organs will be possible.

Several basic research trials deal with the colonization of various cell types on different kinds of scaffolds or extra cellular matrix components for tissue engineering as described by K. Macfelda et al, in their publication, Behavior of cardiomyocytes and skeletal muscle cells on different extracellular matrix components—relevance for cardiac tissue engineering and as described by Santos M I, et al. in their publication, Crosstalk between osteoblasts and endothelial cells co-cultured on a polycaprolactone-starch scaffold and the in vitro development of vascularization and Baino F et al in their publication, Feasibility, tailoring and properties of polyurethane/bioactive glass composite scaffolds for tissue engineering. Engineered components should serve for either the total replacement (e.g. heart valves) respectively for compensation of affected tissue (e.g. nerves) or for implantation of engineered tissue into harmed parts of an organ (e.g. muscles).

The main impact of this target is that in tissue engineering autologous cells (cells from the patient who will receive the transplant) or stem cells are used. Therefore no rejection by immuno reaction occurs. In case of heart valves patients will not have to get anticoagulation therapy as it is necessary when implanting exogenous material. Anticoagulation is associated with severe adverse effects.

Some examples for today's tissue engineering approaches: A. Colonizing cells on various matrices: Heart valves engineering by colonizing endothelial cells on decellularized porcine valves as described by G. Seebacher et al. in their publication, Biomechanical properties of decellularized porcine pulmonary valve conduits. Autologous transplant formation: Some of current regenerative medicine approaches for musculoskeletal tissue repair focuses on the transplantation of in vitro preformed three-dimensional autologous cell/biomaterial composites. E.g. in muscle, heart, bone, cartilage etc. as discussed by Liao H and Zhou G Q in Development and Progress of Engineering of Skeletal Muscle Tissue; M. Sittinger et al. in Tissue engineering and autologous transplant formation: practical approaches with resorbable biomaterials and new cell culture techniques; D. Barnewitz et al. in Treatment of articular cartilage defects in horses with polymer-based cartilage tissue engineering grafts and M. Endres et al. in Osteogenic induction of human bone marrow-derived mesenchymal progenitor cells in novel synthetic polymer-hydrogel matrices. B. Engineering new tissue: Vessels: Tissue-engineered vascular grafts can serve as autologous conduits in bypass surgery or whenever blood supply to a tissue is broken and new vessels are required as described by MP Brennan et al. in Tissue-engineered vascular grafts demonstrate evidence of growth and development when implanted in a juvenile animal model and T R Dunkern et al. in A novel perfusion system for the endothelialisation of PTFE grafts under defined flow. Nerves: Nerve tissue engineering is a promising field for the treatment of several kinds of nerve transection, palsy and even degenerative nerve diseases as described by J D Yuan et al. in Novel three-dimensional nerve tissue engineering scaffolds and its biocompatibility with Schwann cells. Intervertebral discs: Symptomatic intervertebral disc degeneration is associated with several spinal diseases. Tissue engineering provides a promising approach to recover the functionality of the degenerative intervertebral disc as described by Yang X and Li X in Nucleus pulposus tissue engineering: a brief review. Skin transplants: Skin is an important tissue engineering target for reconstructive surgery of burns victims, but increasingly also to assist in the healing of diabetes related ulcers as described by C. Johnen et al. in Culture of subconfluent human fibroblasts and keratinocytes using biodegradable transfer membranes; K A Blackwood et al. in Development of biodegradable electrospun scaffolds for dermal replacement and F. Gossiel et al. in Use of an in vitro model of tissue-engineered skin to investigate the mechanism of skin graft Harrison contraction.

More examples for tissue engineering in reconstructive and regenerative medicine include cornea engineering for replacement due to several kinds of corneal degeneration or trauma, ears or noses engineered out of cartilage cells for replacement or aesthetic surgery purposes and skin flaps and soft tissue for breast reconstruction after amputation due to breast cancer.

Cell activation/treatment: A. Cell multiplication: All kind of cell multiplication or cell activation as it is necessary before several kinds of cell treatments can be done with the water bath device 100, e.g. stem cell therapy or cells before bone marrow transplantation as described by Ikehara S. in The Future of Stem Cell Transplantation in Autoimmune Disease. In experimental studies cells from different tissues are used for transplantation, even in other tissues (e.g. skeletal muscle cells into myocardium) and after transfection of the genome for improvement of specific cell properties as described by S. Aharinejad et al. in Colony-stimulating factor-1 transfection of myoblasts improves the repair of failing myocardium following autologous myoblast transplantation.

B. Stem cell differentiation Today there are some signs that shock waves would be able to promote stem cell differentiation as described by F S Wang et al. in Extracorporeal shock wave promotes growth and differentiation of bone-marrow stromal cells towards osteoprogenitors associated with induction of TGF-beta1 and I. Nasonkin et al. in Long-Term, Stable Differentiation Of Human Embryonic Stem Cell-Derived Neural Precursors Grafted Into The Adult Mammalian Neostriatum.

This could be necessary for several kinds of stem cell treatments and may be done before their administration in-vitro with the water bath device 100. This publication by A. Cargnoni et al., Transplantation of allogeneic and xenogeneic placenta-derived cells reduces bleomycin-induced lung fibrosis and T. Yang et al. in Neurotrophism of bone marrow stromal cells to embryonic stem cells: noncontact induction and transplantation to a mouse ischemic stroke model, shows opportunities in which the device 100 could be used.

C. Blood cells: Blood is a source of important cellular or protein based biological products that are essential in the treatment of various bleeding, immunological, and metabolic disorders. The activation of platelets leads to the release of several cytokines and growth factors as described by C Y Su et al. in In vitro release of growth factors from platelet-rich fibrin (PRF): a proposal to optimize the clinical applications of PRF. Therefore there is an increasing interest in the use of platelet-rich fractions as a therapeutic source of growth factors in regenerative medicine as described by S. Kazemnejad et al. in Efficient replacing of fetal bovine serum with human platelet releasate during propagation and differentiation of human bone-marrow-derived mesenchymal stem cells to functional hepatocyte-like cells. The activity of blood cells and their ability to release growth factors could be advanced by shock wave therapy. The use of blood cells for transfusion following immunoglobulin or antibody deficiency is discussed as described by T. Burnouf et al. in Comparative removal of solvent and detergent viral inactivating agents from human intravenous immunoglobulin G preparations using SDR HyperD and C18 sorbents.

Cell membrane permeation: Making any kind of agent and pharmaceuticals permeate through cell membranes into cells (even in germ-buds) for labelling or therapeutic reasons could be performed by shock wave therapy as it is known that shock waves alter cell membrane permeability and cause hyper polarisation as described by F S Wang et al. in Physical shock wave mediates membrane hyperpolarization and Ras activation for osteogenesis in human bone marrow stromal cells. There are several reasons for making agents permeate into cells. E.g. (Adeno-) Viruses and plasmids are used for genome transfection. Agents against cancer are attempted to get inserted into malign cells, Example: Ribosome inactivating proteins by RNA knockdown in cancer cells as described by Michael Delius and Gerhard Adams in Shock Wave Permeabilization with Ribosome Inactivating Proteins: A New Approach to Tumor Therapy; W. Shen et al. in Oncolytic adenovirus mediated Survivin knockdown by RNA interference suppresses human colorectal carcinoma growth in vitro and in vivo and S. Aharinejad et al. in Colony-stimulating factor-1 transfection of myoblasts improves the repair of failing myocardium following autologous myoblast transplantation. All of the above research could be more effectively achieved with the use of the device 100.

Other sizes and shapes of the device 100, especially expansion of the length for further propagation of shock waves can be done. The device can be filled with fluids other than water. For example, the device 100 can be filled with blood for activation of blood cells for transfusion. Cells or tissue not in flasks or other fluid filled tanks can be dunked or submerged into the container of the device 100. The container of the device 100 can be filled with any kind of sterile culture medium and cells or tissue cultured directly therein. Shock waves can not only be applied in a horizontal manner as shown, but also vertically or in any direction, even moving around the sample. Treatments can not only by the use of shock waves can be preformed, but also other kinds of sound waves such as ultrasound or light of different frequencies or magnetism can be used with the device 100. The device 100 can be used with or without a heater, even with cooling by way of example with the use of a Peltier cooling plate if so desired. Absorbers can be provided on the posterior wall of the container/or other walls as well as the bottom and a top wall if so desired. Different shock wave sources focused, unfocused, planar, spherical and different placings of the flask in the field and different positioning and distances leading to dose/dosis variations are possible with the use of the device 100.

All these and other variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A device for treating cells or cultures of cells comprises;
a fluid holding tank, having sides or walls and a bottom, with an opening in a wall or side for receiving an applicator for transmitting energy into a holding tank.

2. The device of claim 1 further comprises:
an adapter for insertion into the opening, the adapter being for attachment to the applicator for transmitting energy into holding tank, the adapter forms a fluid tight seal between the opening and the adapter.

3. The device of claim 2 wherein the adapter receives and holds a lens or diaphragm through which the transmission as energy in the form of acoustic waves passes.

4. The device of claim 1 further comprises:
a cell culture flask holding mechanism to which a cell culture flask can be secured.

5. The device of claim 4 wherein the cell culture flask holding mechanism includes a slotted bar; a flask holder and a locking screw wherein the screw holding a flask can be moved in the slot to a predetermined distance fixing the distance between the flask and the opening.

6. The device of claim 1 further comprises:
a heater for heating fluid in the holding tank.

7. The device of claim 6 further comprises:
a temperature sensor for measuring the fluid temperature in the holding tank.

8. The device of claim 7 further comprises:
a power supply to power the heater; and
a temperature controller connected to the power supply, heater and sensor to regulate a fluid temperature in the holding tank.

9. The device of claim 5 wherein the flask holder has a locking screw to secure a flask in the holder.

10. The device of claim 5 wherein the bar has a scale adjacent the slot to indicate the distance between a front face of a held flask and the opening.

11. The device of claim 2 further comprises:
a modified applicator attached to the adapter, the modified applicator transmits acoustic shock waves into the holding tank to stimulate cells being cultured in a flask held in the flask holding mechanism.

12. The device of claim 2 further comprises:
a modified applicator attached to the adapter, the modified applicator transmits ultrasonic sound waves into the holding tank to stimulate cells being cultured in a flask held in the flask holding mechanism.

13. The device of claim 2 further comprises:
a modified applicator attached to the adapter, the modified applicator transmits magnetic pulses into the holding tank to stimulate cells being cultured in a flask held in the flask holding mechanism.

14. The device of claim 11 wherein the acoustic shock waves are focused waves.

15. The device of claim 11 wherein the acoustic shock waves are unfocused waves.

16. The device of claim 15 wherein the acoustic shock waves are low energy shock waves.

17. A method of treating cells comprises the steps of:
placing a flask containing cells in a fluid filled holding tank having an opening for receiving an applicator for transmitting energy waves or pulses; and
activating the applicator transmitting energy to the flask to stimulate the cells.

18. The method of claim 17 further comprises:
fixing the distance between the flask and the applicator to a predetermined distance prior to transmitting energy.

19. The method of claim 17 further comprises:
controlling the temperature of the fluid within the fluid filled holding tank.
